(19) United States
(12) Patent Application Publication
(10) Pub. No.: US 2023/0414781 A1
(43) Pub. Date: Dec. 28, 2023

(54) ANTI-CD276 ANTIBODY, ANTIBODY-DRUG CONJUGATE, AND USE THEREOF

(71) Applicant: BLISS BIOPHARMACEUTICAL (HANGZHOU) CO., LTD., Hangzhou, Zhejiang (CN)

(72) Inventors: Bing XIA, Hangzhou, Zhejiang (CN); Hao KONG, Hangzhou, Zhejiang (CN); Lixia CAO, Hangzhou, Zhejiang (CN); Fangdun JIANG, Hangzhou, Zhejiang (CN); Yuhong ZHOU, Hangzhou, Zhejiang (CN); Ziping WEI, Hangzhou, Zhejiang (CN)

(21) Appl. No.: 18/253,695

(22) PCT Filed: Nov. 19, 2021
(86) PCT No.: PCT/CN2021/131785

§ 371 Date: May 19, 2023

(30) Foreign Application Priority Data
Nov. 20, 2020 (WO) PCT/CN2020/130409

(51) Int. Cl. A61K 47/68; C07K 16/28; C07K 16/32; A61P 35/00

(52) U.S. Cl. CPC A61K 47/6889; A61K 47/6851; A61K 47/6849; A61P 35/00; C07K 16/2863; C07K 16/32; A61K 47/6803

(57) ABSTRACT

Isolated anti-human B7-H3 antibody includes two heavy chains including a hinge region comprising an amino acid sequence that allows site-specific conjugation of cytotoxic drugs. Each heavy chain includes a human CH1 domain located upstream of and connected to the hinge region. The CH1 domain comprises a cysteine at the position of 142 according to the IMGT numbering scheme. ADCs containing the antibody conjugated with a cytotoxic drug are also provided. Pharmaceutical compositions including the antibody or the ADCs, and methods of treating cancer using the pharmaceutical compositions are also provided.

### CROSS REFERENCE TO RELATED APPLICATION

This application claims priority to International Application No. PCT/CN2020/130409 filed Nov. 20, 2020, the disclosure of which is incorporated by reference herein in its entirety.

### BACKGROUND

CD276, also known as B7 homologue 3 (B7-H3), shares up to 30% amino acid identity with other B7 family members^{[1]}. B7-H3 protein is involved in the regulation of proliferation, apoptosis, invasion, cell cycle, cell differentiation, cell autophagy, and epithelial-mesenchymal transition. It is present at low levels in most normal tissues but is overexpressed in a wide variety of cancers, including bladder, breast, cervical, colorectal, esophageal, glioma, kidney, liver, lung, ovarian, pancreatic, prostate, intrahepatic cholangiocarcinoma, oral squamous cell carcinoma, endometrial cancer, and melanoma^{[2-6]}. Moreover, B7-H3 protein is highly expressed in tumor vessels of human lung, breast, colon, endometrial, renal and ovarian cancer, but not in the angiogenic vessels of normal ovary^{[7-9]}. Thus, B7-H3 directed therapeutic agents may have a higher degree of specificity for tumor vessels than current anti-angiogenic agents that cannot distinguish physiological and pathological angiogenesis. B7-H3 overexpression by tumor cells and tumor endothelial cells (TECs) makes it an appealing target for the development of therapeutic agents to simultaneously destroy both cell types.

An Fc-enhanced humanized anti-CD276 antibody, enoblituzumab, has shown activities delaying the growth of a variety of primary tumor types in preclinical studies and has advanced to Phase I clinical trials. It was investigated in treating refractory B7-H3-expressing tumors such as melanoma (NCT01391143), and B7-H3-expressing neoplasms including osteosarcoma and Ewing's sarcoma (NCT02982941). MGA271 is another B7-H3-targeting antibody that exhibited potent antitumor activity in xenograft models of B7-H3-expressing renal cell and bladder carcinoma^{[10]}. 8H9, a humanized antibody originally identified based on its selective reactivity with human tumors cells, was later found to recognize CD276, is also in Phase I clinical trials^{[11, 12]}. Humanized 8H9 antibodies could regulate the inhibitory immune properties of B7-H3 on target tumors and affect the immune checkpoint blockade^{[13]}.

ADCs are monoclonal antibodies conjugated with cytotoxic agents. They take advantage of target specificities to tumor cell-surface proteins and deliver toxic payload with high potencies to tumors with specificity and potency not achievable with traditional chemotherapies. Currently several ADCs targeting B7-H3 are at different stages of preclinical and clinical development. It has been demonstrated in multiple preclinical models that B7-H3-targeting ADCs were able to eradicate established tumors and improve overall survival significantly, suggesting the therapeutic potentials of these molecules^{[1]}. For example, exatecan derivative (DX-8951 derivative, DXd), used for drug conjugation as DXd-ADC targeting B7-H3, showed effective antitumor efficacy as well as less adverse effects. It is now being evaluated in multiple Phase II clinical trials^{[14]}. 131I-labeled anti-B7-H3 mAb (131I-4H7) is a radiobiological agent and had treatment effects on nude mice with human RCC, and has been advanced to Phase III clinical trials^{[15]}.

### SUMMARY OF THE INVENTION

In one aspect, the present invention provides an isolated antibody, or an antigen-binding portion thereof, comprising: two heavy chains each comprising: (a1) a heavy chain hinge region comprising the amino acid sequence set forth in any of SEQ ID NOs: 12-24; (a2) a heavy chain variable domain comprising a CDR1 region, a CDR2 region, and a CDR3 region comprising the amino acid sequences of SEQ ID NO: 6, SEQ ID NO: 7, and SEQ ID NO: 8, respectively, and two light chains each comprising (a3) a light chain variable domain comprising a CDR1 region, a CDR2 region, and a CDR3 region comprising the amino acid sequences of SEQ ID NO: 9, WAS, and SEQ ID NO: 10, respectively. The antibody can specifically bind to human B7-H3 protein. In some embodiments, the amino acid sequence comprised in the heavy chain hinge region is SEQ ID NO: 12. In some embodiments, each of the heavy chains further comprises: a human CH1 domain located upstream of and connected to the hinge region, the CH1 domain comprising a cysteine at the position of 142 according to the IMGT numbering scheme.

In another aspect, the present invention provides an isolated antibody, or an antigen-binding portion thereof, comprising: two heavy chains each comprising: (a) a hinge region comprising an amino acid sequence of: —(X₁)—C—(X₂)—CPPCP—, wherein X₁ is a polypeptide segment having 0-7 amino acid residues each independently selected from any amino acid residue that is not a cysteine residue, and X₂ is a polypeptide segment having 2-7 amino acid residues each independently selected from any amino acid residue that is not a cysteine residue; (b) a human CH1 domain located upstream of and connected to the hinge region, the CH1 domain comprising a cysteine at the position of 142 according to the IMGT numbering scheme, wherein the antibody specifically binds to human B7-H3 protein. In some embodiments, the amino acid sequence of comprised in the hinge region is selected from the group consisting of SEQ ID NOs: 11-24, for example SEQ ID NO: 11, SEQ ID NO: 12. In some embodiments, the antibody includes a heavy chain variable domain comprising a CDR1 region, a CDR2 region, and a CDR3 region comprising the amino acid sequences of SEQ ID NO: 6, SEQ ID NO: 7, and SEQ ID NO: 8, respectively, and a light chain variable domain comprising a CDR1 region, a CDR2 region, and a CDR3 region comprising the amino acid sequences of SEQ ID NO: 9, WAS, and SEQ ID NO: 10, respectively.

In some embodiments, the antibody described herein can further comprising two kappa (κ) light chains each paired with one of the heavy chains. The two heavy chains in the antibody can be identical. The two light chains in the antibody can be identical.

In some embodiments, the CH1 domain of the antibody has the same sequence as that of the CH1 domain of a native human IgG2, IgG3, or IgG4 subclass antibody, or the sequence of that of the CH1 domain of a native human IgG1 antibody with the mutation S142C.

In some embodiments, each of the heavy chains can further comprise an Fc domain of a native human IgG1, IgG2, IgG3, IgG4 subclass antibody downstream of and connected to the hinge region, wherein the Fc domain optionally includes one or more substitutions.

In some embodiments, each of the heavy chains comprises a variable domain comprising the amino acid sequence set forth in SEQ ID NO: 1. In some embodiments, each of the heavy chains comprises an amino acid sequence set forth in SEQ ID NO: 4.

In some embodiments, each of the light chains comprises variable domain comprising the amino acid sequence set forth in SEQ ID NO: 2. In some embodiments, each of the light chains comprises an amino acid sequence set forth in SEQ ID NO: 5.

The antibodies of the present invention can be monoclonal antibodies, and they can be camelid, chimeric, human or humanized.

In a further aspect, an antibody-drug conjugate (ADC) or a pharmaceutically acceptable salt thereof, are provided, which includes an antibody described herein, which is conjugated with a cytotoxic drug and a chemical linker.

In one embodiment, the ADC can include: (A) an isolated antibody, or an antigen-binding portion thereof, comprising: (a1) a heavy chain hinge region comprising the amino acid sequence set forth in SEQ ID NO: 11; (a2) a heavy chain variable domain comprising a CDR1 region, a CDR2 region, and a CDR3 region comprising the amino acid sequences of SEQ ID NO: 6, SEQ ID NO: 7, and SEQ ID NO: 8, respectively, and (a3) a light chain variable domain comprising a CDR1 region, a CDR2 region, and a CDR3 region comprising the amino acid sequences of SEQ ID NO: 9, WAS, and SEQ ID NO: 10, respectively; (B) a cytotoxic drug,
wherein the isolated antibody or an antigen-binding portion thereof is conjugated to the cytotoxic drug by a chemical linker.

The cytotoxic drug of the ADC can be selected from the group consisting of eribulin, monomethyl auristatin E (MMAE), monomethyl auristatin F (MMAF), auristatin E, auristatin F, maytansine DM1 and DM4, maytansinol, sandramycin, pyrrolobenzodiazepine, pyrrolobenzodiazepine dimer, anthracyclines, calicheamicin, dolastatin 10, duocarmycin, doxorubicin, thailanstatin A, uncialamycin, amanitins, ricin, diphtheria toxin, ¹³¹I, interleukins, tumor necrosis factors, chemokines, irinotecan (SN38), exatecan, and nanoparticles. In some embodiments, the chemical linker can comprise a portion that is selected from the group consisting of 6-maleimidocaproyl (MC), maleimidopropionyl (MP), valine-citrulline (Val-Cit), alanine-phenylalanine (ala-phe), p-aminobenzyloxycarbonyl (PAB), 6-maleimidocaproyl-Val-Cit-p-aminobenzyloxycarbonyl (MC-Val-Cit-PAB), Mal-PEGⁿ-Val-Cit-PAB (n=1-20), Mal-amido-PEGₙ-Val-Cit-PAB (n=1-20), MC-Gly-Gly-Phe-Gly, Phe-Lys(Fmoc)-PAB, Aloc-D-Ala-Phe-Lys(Aloc)-PAB-PNP, Boc-Phe-(Alloc)Lys-PAB-PNP, and perfluorophenyl 3-(pyridine-2-yldisulfanyl) propanoate. In an example, the cytotoxic drug is eribulin. In another example, the cytotoxic drug is MMAE.

In a further aspect, the present invention provides a pharmaceutical composition comprising: an isolated antibody or an antigen binding portion thereof, or an ADC of pharmaceutically acceptable salt thereof, as described herein, and (b) a pharmaceutical acceptable carrier.

In a further aspect, the present invention provides a method of treating cancer in a human subject, comprising administering an effective amount of the pharmaceutical composition herein. The cancer can be a cancer associated with overexpression of B7-H3 protein. For example, the cancer can be selected from the group consisting of a cancer of the head and neck, kidney, skin, colon, glioblastoma, glioma, thyroid, mesothelioma, melanoma, pancreas, lung, breast, ovary, prostate, and bladder.

### BRIEF DESCRIPTION OF THE DRAWINGS

FIG. 1 shows schematic diagrams of the amino acid numbering system of antibodies as used in this application. (a) IMGT-based amino acid numbering scheme of human IgG1(u). (b) the numbering scheme for mutants (for the IgG1 hinge region).

FIG. 2 shows the physicochemical testing results of 27B4H4L3 and BH73 antibodies. SDS-PAGE analysis of reducing (R) and non-reducing (NR) antibodies: 27B4H4L3 antibody (a) and BH73 antibody (b); purity and yields of the antibodies produced in HEK293 cells (c); SEC-HPLC analysis of purified antibodies, 27B4H4L3 antibody (d) and BH73 antibody (e).

FIG. 3 shows conjugation profiles of ADCs made of 27B4H4L3 and BH73 antibodies. HIC profiles of ADCs made of 27B4H4L3 antibody and BH73 antibody: (a) 27B4H4L3-MMAE (b), BH73-MMAE.

FIG. 4 shows binding curves of 27B4H4L3 antibody and 27B4H4L3-MMAE to recombinant B7-H3 protein.

FIG. 5 shows cytotoxicity curves of the 27B4H4L3-MMAE, BH73-MMAE and BR0102-DXd to B7-H3-expressing cancer cells: (a) U87, (b) BT-474, (c) HT-29 and (d) A431.

FIG. 6 shows in vivo efficacy of BH73-MMAE and BR0102-DXd against colorectal cancer HT29 xenografts in athymic nude mice.

### DETAILED DESCRIPTION

The present disclosure provides antibodies and ADCs targeting B7-H3 using anti-B7-H3 antibody and demonstrated their potent antitumor capabilities.

In one aspect, the present invention provides an isolated antibody, or an antigen-binding portion thereof, comprising:
two heavy chains each comprising:
(a1) a heavy chain hinge region comprising the amino acid sequence set forth in any of SEQ ID NOs: 12-24;
(a2) a heavy chain variable domain comprising a CDR1 region, a CDR2 region, and a CDR3 region comprising the amino acid sequences of SEQ ID NO: 6, SEQ ID NO: 7, and SEQ ID NO: 8, respectively, and
two light chains each comprising:
(a3) a light chain variable domain comprising a CDR1 region, a CDR2 region, and a CDR3 region comprising the amino acid sequences of SEQ ID NO: 9, WAS, and SEQ ID NO: 10, respectively.

The antibody can specifically bind to human B7-H3 protein. In some embodiments, the amino acid sequence comprised in the heavy chain hinge region is SEQ ID NO: 12. In some embodiments, each of the heavy chains further comprises: a human CH1 domain located upstream of and connected to the hinge region, the CH1 domain comprising a cysteine at the position of 142 according to the IMGT numbering scheme.

Antibodies containing heavy chain in this format preferably form H-L inter-chain disulfide bond between C142 (or a cysteine near 142^{th} position, according to the IMGT numbering system as further described below) of the CH1 domain with the last cysteine residue in the light chain. The cysteine residue in the hinge region upstream of the native CPPCP sequence forms a third H—H inter-chain disulfide bond. The cysteine at or near amino acid 142 in the CH1 domain could be introduced by mutation or insertion of a single amino acid in IgG1 subtype, or could come from the natural cysteine residue in the CH1 domain of IgG2, IgG3, or IgG4 subtypes. Compared with native H-L disulfide bonds in IgG1, which are between a cysteine in the hinge region of the IgG1 antibody heavy chain and the terminal end of the paired light chain, the H-L disulfide bond in this format is more stable and can be kept intact in the reducing condition during the drug conjugation to the antibody. This dramatically reduces the chances of obtaining ADCs which contains light chain drug conjugates.

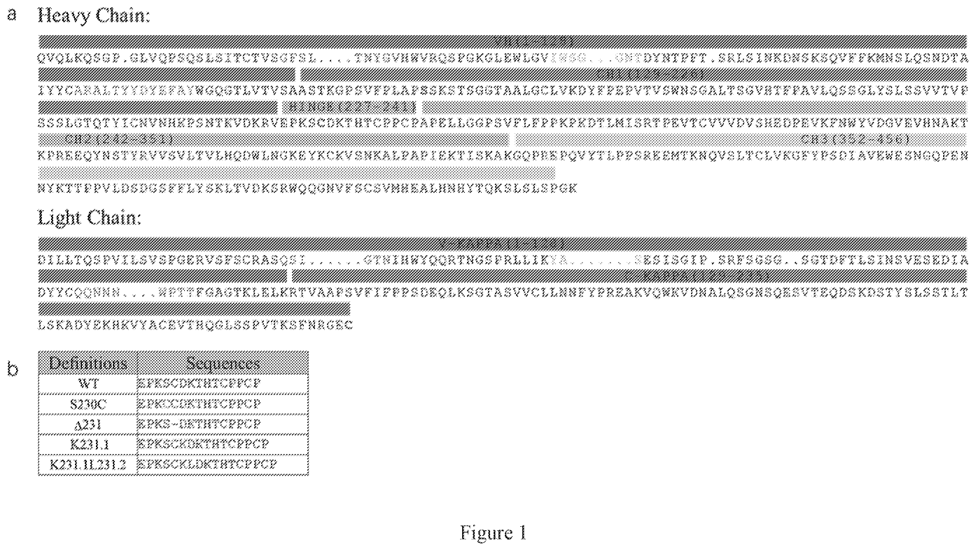

The IMGT numbering system for immunoglobulin superfamily is used herein to simplify the numbering scheme (schematic diagram in FIG. 1a), where the VH or VL domain each contains amino acid residues 1-128. Accordingly, amino acids in the CH1 domain are numbered as aa129-226; x domain as aa129-235; hinge region as aa227-241 (according to IgG1); CH2 as aa242-351, and CH3 as aa352-456. Based on this numbering scheme, the H-L inter-chain disulfide bond in wild-type IgG1(κ) would be formed between H(C231)-L(C235), while in IgG2(κ)(or IgG3(κ) or IgG4(κ)) it could be formed between H(C142)-L(C235). IgG1 mutant with heavy chain serein 230 changed to cysteine would be named IgG1 (S230C), while with deletion of C231 would be named IgG1(Δ231). Insertion of a lysine after C231 would be named K231.1, and insertion of two amino acids, KL, after C231 would be named K231.1 L231.2 (see FIG. 1b which shows a few examples of notations for mutations introduced in the hinge region of the IgG1). For example, the hinge region of a control/reference antibody 27B4H4L3 comprises the amino acid sequence of EPKSCDKTHTCPPCP (SEQ ID NO: 11), and an example of the engineered antibodies of the present invention, clone BH73, includes a modified hinge amino acid sequence comprising EPPKSDCKTKTVECPPCP (SEQ ID NO: 12). Note that the control antibody 27B4H4L3 includes a human IgG1 CH1, whereas antibody BH73 contains a human IgG2 CH1.

In another aspect, the present invention provides an isolated antibody, or an antigen-binding portion thereof, comprising: two heavy chains each comprising: (a) a hinge region comprising an amino acid sequence of: —(X₁)—C—(X₂)—CPPCP—, wherein X₁ is a polypeptide segment having 0-7 amino acid residues each independently selected from any amino acid residue that is not a cysteine residue, and X₂ is a polypeptide segment having 2-7 amino acid residues each independently selected from any amino acid residue that is not a cysteine residue; (b) a human CH1 domain located upstream of and connected to the hinge region, the CH1 domain comprising a cysteine at the position of 142 according to the IMGT numbering scheme, wherein the antibody specifically binds to human B7-H3 protein.

In some embodiments, the amino acid sequence of comprised in the hinge region is selected from the group consisting of SEQ ID NOs: 11-24, for example SEQ ID NO: 11, SEQ ID NO: 12. In some embodiments, the antibody described herein can further comprising two K light chains each paired with one of the heavy chains. The two heavy chains in the antibody can be identical. The two light chains in the antibody can be identical.

In some embodiments, the CH1 domain of the antibody has the same sequence as that of the CH1 domain of a native human IgG2, IgG3, or IgG4 subclass antibody, or the sequence of that of the CH1 domain of a native human IgG1 antibody with the mutation S142C.

In some embodiments, each of the heavy chains can further comprise an Fc domain of a native human IgG1, IgG2, IgG3, IgG4 subclass antibody downstream of and connected to the hinge region, wherein the Fc domain optionally includes one or more substitutions.

In some embodiments, each of the heavy chains comprises a variable domain comprising the amino acid sequence set forth in SEQ ID NO: 1. In some embodiments, each of the heavy chains comprises an amino acid sequence set forth in SEQ ID NO: 4.

In some embodiments, each of the light chains comprises variable domain comprising the amino acid sequence set forth in SEQ ID NO: 2. In some embodiments, each of the light chains comprises an amino acid sequence set forth in SEQ ID NO: 5.

In some embodiments, each of the heavy chains of the antibody comprises a variable domain comprising the amino acid sequence set forth in SEQ ID NO: 1, and each of the light chains of the antibody comprises a variable domain comprising the amino acid sequence set forth in SEQ ID NO: 2.

In some embodiments, the antibody comprises a heavy chain comprising the amino acid sequence set forth in SEQ ID NO: 4 and a light chain comprising the amino acid sequence set forth in SEQ ID NO:5.

The term “isolated antibody” as used herein refers to an antibody that is substantially free of other antibodies having different antigenic specificities. An isolated antibody that specifically binds to an antigen is substantially free of antibodies that do not bind to that antigen.

The term “monoclonal antibody” as used herein refer to a preparation of a population of antibody molecules of substantially homogeneous molecular composition, wherein the individual antibodies in the population of the antibody molecules are identical except for possible naturally occurring mutations that may be present in miniscule amounts.

An antibody or molecule that “specifically binds to human B7-H3” refers to an antibody or polypeptide molecule that binds to human B7-H3 protein but does not substantially bind to proteins that are not human B7-H3 proteins.

The CDRs (complementarity determining regions) of an antibody are defined by those skilled in the art using a variety of methods/systems. These systems and/or definitions have been developed and refined over a number of years and include Kabat, Chothia, IMGT, AbM, and Contact. The Kabat definition is based on sequence variability and is commonly used. The Chothia definition is based on the location of the structural loop regions. The IMGT system is based on sequence variability and location within the structure of the variable domain. The AbM definition is a compromise between Kabat and Chothia. The Contact definition is based on analyses of the available antibody crystal structures. An Exemplary system is a combination of Kabat and Chothia.

DNA encoding an amino acid sequence variant of a starting polypeptide can prepared by a variety of methods known in the art. These methods include, but are not limited to, preparation by site-directed (or oligonucleotide-mediated) mutagenesis, PCR mutagenesis, and cassette mutagenesis of an earlier prepared DNA encoding the polypeptide. Variants of recombinant antibodies may be constructed also by restriction fragment manipulation or by overlap extension PCR with synthetic oligonucleotides. Mutagenic primers encode the cysteine codon replacement(s). Standard mutagenesis techniques can be employed to generate DNA encoding such mutant engineered antibodies.

In yet a further aspect, the present disclosure provides a nucleic acid molecule encoding the antibody or antigen-binding portion thereof of any of the antibody described herein. A host cell (e.g., a CHO cell, a lymphocytic cell, a human embryonic kidney cell, or microorganisms, such as E. coli and fungi, such as yeast) containing an expression vector containing the nucleic acid molecule, can be used to produce antibodies of the present disclosure, preferably monoclonal antibodies. In one embodiment, DNA encoding partial or full-length antibody of the present disclosure can be obtained by standard molecular biology techniques is inserted into one or more expression vectors such that the genes are operatively linked to transcriptional and translational regulatory sequences. The term “operatively linked” is intended to mean that an antibody gene is ligated into a vector such that transcriptional and translational control sequences within the vector serve their intended function of regulating the transcription and translation of the antibody gene. The term “regulatory sequence” is intended to include promoters, enhancers and other expression control elements (e.g., polyadenylation signals) that control the transcription or translation of the antibody genes. Such regulatory sequences are described, e.g., in Goeddel (Gene Expression Technology. Methods in Enzymology 185, Academic Press, San Diego, Calif. (1990)). Preferred regulatory sequences for mammalian host cell expression include viral elements that direct high levels of protein expression in mammalian cells, such as promoters and/or enhancers derived from cytomegalovirus (CMV), Simian Virus 40 (SV40), adenovirus, e.g., the adenovirus major late promoter (AdMLP) and polyoma. Alternatively, nonviral regulatory sequences can be used, such as the ubiquitin promoter or β-globin promoter. Still further, regulatory elements composed of sequences from different sources, such as the SRα promoter system, which contains sequences from the SV40 early promoter and the long terminal repeat of human T cell leukemia virus type 1 (Takebe et al., (1988) Mol. Cell. Biol. 8:466-472). The expression vector and expression control sequences are chosen to be compatible with the expression host cell used.

The antibody encoding DNA can be inserted into the expression vector. The recombinant expression vector can encode a signal peptide that facilitates secretion of the antibody chain from a host cell. The antibody encoding DNA can be cloned into the vector such that the signal peptide is linked in-frame to the amino terminus of the antibody encoding DNA. The signal peptide can be an immunoglobulin signal peptide or a heterologous signal peptide (i.e., a signal peptide from a non-immunoglobulin protein).

In a further aspect, an antibody-drug conjugate (ADC) or a pharmaceutically acceptable salt thereof, is provided. In some embodiments, the ADC comprises an antibody of the present disclosure as described herein, conjugated to a cytotoxic drug by a chemical linker.

In some embodiments, the antibody portion of the ADC includes: (a1) a heavy chain hinge region comprising the amino acid sequence set forth in any of SEQ ID NOs: 12-24; (a2) a heavy chain variable domain comprising a CDR1 region, a CDR2 region, and a CDR3 region comprising the amino acid sequences of SEQ ID NO: 6, SEQ ID NO: 7, and SEQ ID NO: 8, respectively; (a3) each of the heavy chains further comprises: a human CH1 domain located upstream of and connected to the hinge region, the CH1 domain comprising a cysteine at the position of 142 according to the IMGT numbering scheme and (a4) a light chain variable domain comprising a CDR1 region, a CDR2 region, and a CDR3 region comprising the amino acid sequences of SEQ ID NO: 9, WAS, and SEQ ID NO: 10, respectively. In one example, the antibody portion of the ADC is BH73 antibody.

In some embodiments, the ADC includes (A) an isolated antibody, or an antigen-binding portion thereof, comprising: (a1) a heavy chain hinge region comprising the amino acid sequence set forth in SEQ ID NO: 11; and (a2) a heavy chain variable domain comprising a CDR1 region, a CDR2 region, and a CDR3 region comprising the amino acid sequences of SEQ ID NO: 6, SEQ ID NO: 7, and SEQ ID NO: 8, respectively, and (a3) a light chain variable domain comprising a CDR1 region, a CDR2 region, and a CDR3 region comprising the amino acid sequences of SEQ ID NO: 9, WAS, and SEQ ID NO: 10, respectively; and (B) a cytotoxic drug,
wherein the isolated antibody or an antigen-binding portion thereof is conjugated to the cytotoxic drug by a chemical linker. In one example, the antibody portion of the ADC is 27B4H4L3.

In some embodiments, the cytotoxic drug can be selected from the group consisting of eribulin, monomethyl auristatin E (MMAE), monomethyl auristatin F (MMAF), auristatin E, auristatin F, maytansine DM1 and DM4, maytansinol, sandramycin, pyrrolobenzodiazepine, pyrrolobenzodiazepine dimer, anthracyclines, calicheamicin, dolastatin 10, duocarmycin, doxorubicin, thailanstatin A, uncialamycin, amanitins, ricin, diphtheria toxin, ¹³¹I, interleukins, tumor necrosis factors, chemokines, irinotecan (SN38), exatecan, and nanoparticles. In specific embodiments, the cytotoxic drug in the ADC is eribulin. In other embodiments, the cytotoxic drug in the ADC is MMAE.

The chemical linker linking the antibody portion and the cytotoxic drug can be cleavable or non-cleavable. In some embodiments, the linker comprises a PEGn spacer where n is between 1 and 20 (i.e., having 1 to 20 repeat units (CH2CH₂O)). In some embodiments, the chemical linker further comprises a linker segment connected to the PEGn spacer. In some embodiments, the chemical linker comprises a linker segment but does not comprise a PEGn spacer. In some embodiments, the chemical linker can include a segment that is selected from the group consisting of 6-maleimidocaproyl (MC), maleimidopropionyl (MP), valine-citrulline (Val-Cit), alanine-phenylalanine (ala-phe), p-aminobenzyloxycarbonyl (PAB), 6-maleimidocaproyl-Val-Cit-p-aminobenzyloxycarbonyl (MC-Val-Cit-PAB), Mal-PEGₙ-Val-Cit-PAB (n=1-20), Mal-amido-PEGₙ-Val-Cit-PAB (n=1-20), MC-Gly-Gly-Phe-Gly, Phe-Lys(Fmoc)-PAB, Aloc-D-Ala-Phe-Lys(Aloc)-PAB-PNP, Boc-Phe-(Alloc)Lys-PAB-PNP, and perfluorophenyl 3-(pyridine-2-yldisulfanyl) propanoate, or combinations thereof.

In the present disclosure, the pharmaceutically acceptable salts of the ADCs include acid addition salts of inorganic acids, carboxylic acids and sulfonic acids, for example, salts of the following acids: hydrochloric acid, hydrobromic acid, sulfuric acid, phosphoric acid, methanesulfonic acid, ethanesulfonic acid, benzenesulfonic acid, toluenesulfonic acid, naphthalene disulfonic acid, acetic acid, trifluoroacetic acid, propionic acid, lactic acid, tartaric acid, malic acid, citric acid, fumaric acid, maleic acid and benzoic acid.

The pharmaceutically acceptable salts of the antibody-drug conjugates of the present disclosure also include salts of conventional bases, for example alkali metal salts (e.g., sodium salts and potassium salts), alkaline earth metal salts (e.g., calcium salts and magnesium salts) and ammonium salts derived from ammonia or organic amines containing from 1 to 16 carbon atoms, in which the organic amines are, for example, ethylamine, diethylamine, triethylamine, ethyl diisopropylamine, monoethanolamine, diethanolamine, triethanolamine, dicyclohexylamine, dimethylaminoethanol, procaine, dibenzamide, N-methylpiperidine, N-methylmorpholine, arginine, lysine and 1,2-ethylenediamine.

It is understood that an ADC as used herein refers to a molecule that contains both a drug molecule and an antibody (or an antigen binding portion thereof) where the drug and the antibody (or the antigen binding portion thereof) is covalently connected by a linker. An “ADC preparation” herein refers to a collection or population of ADC molecules whose structure may differ due to possibly different attachment sites of the chemical linker to the antibody (or the antigen binding portion thereof). In some embodiments, the chemical linker is primarily or predominantly (e.g., ≥80%, ≥85%, ≥90%, ≥95% or ≥98%) conjugated with cysteines on a heavy chain, resulting in an ADC preparation that is substantially devoid of light chain conjugation. In some embodiments, the chemical linker is conjugated with the antibody predominantly through the cysteines in the hinge region of the heavy chains of the antibody. And in certain embodiments, ADC molecules having drug to antibody ratio (DAR) of 2 accounts for at least 60%, at least 70%, at least 80%, at least 85%, or at least 90% of the total amount of ADC molecules.

In further aspect, the present disclosure provides a pharmaceutical composition comprising one or more antibodies, ADCs or the pharmaceutically acceptable salts thereof, of the present invention, together with a pharmaceutically acceptable carrier. As used herein, “pharmaceutically acceptable carrier” includes pharmaceutically acceptable carriers, excipients or stabilizers. These include but are not limited solvents, dispersion media, coatings, antibacterial and antifungal agents, isotonic and absorption delaying agents, surface active agents, thickening or emulsifying agents, solid binders, dispersion or suspension aids, solubilizers, colorants, flavoring agents, coatings, disintegrating agents, lubricants, sweeteners, preservatives, isotonic agents, and the like that are physiologically compatible. The selection of suitable carrier is within the knowledge of an artisan skilled in the art.

The composition may comprise one or more additional pharmaceutically active ingredients, such as another antibody, a drug, e.g., a cytotoxic or anti-tumor agent. The pharmaceutical compositions of the invention also can be administered in a combination therapy with, for example, another anti-cancer agent, another anti-inflammatory agent, etc.

The pharmaceutical composition can be suitable for intravenous, intramuscular, subcutaneous, parenteral, epidermal, and other routes of administration. Depending on the route of administration, the active ingredient can be coated with a material or otherwise loaded in a material or structure to protect it from the action of acids and other natural conditions that may inactivate it. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Alternatively, the composition of the invention can be administered via a non-parenteral route, such as a topical, epidermal or mucosal route of administration, e.g., intranasally, orally, vaginally, rectally, sublingually or topically.

In a further aspect, the present invention provides a method of treating cancer in a human subject, comprising administering an effective amount of the pharmaceutical composition herein. The cancer can be a cancer associated with overexpression of B7-H3 protein. For example, the cancer can be selected from the group consisting of a cancer of the head and neck, kidney, skin, colon, glioblastoma, glioma, thyroid, mesothelioma, melanoma, pancreas, lung, breast, ovary, prostate, and bladder.

In the administration of the composition to the subject, dosage regimens can be adjusted to provide the optimum desired response (e.g., a therapeutic response). Single bolus or divided doses can be administered based on the subject, the disease to be treated, etc. Dosage unit form as used herein refers to physically discrete units suited as unitary dosages for the subjects to be treated. Each unit contains a predetermined quantity of active ingredient calculated to produce the desired therapeutic effect in association with the required pharmaceutical carrier. Sustained release formulation can be used in which case less frequent administration is required.

For administration of an antibody or ADC pharmaceutical salts thereof of the present disclosure, the dosage may range from about 0.0001 to 100 mg/kg, and more usually 0.01 to 10 mg/kg, of the body weight of the subject. For example, dosages can be 0.3 mg/kg body weight, 1 mg/kg body weight, 3 mg/kg body weight, 5 mg/kg body weight or 10 mg/kg body weight or within the range of 1-10 mg/kg. A suitable treatment regime can be once per week, once every two weeks, once every three weeks, once every four weeks, once a month, etc. Example dosage regimens for an anti-B7-H3 antibody of the invention can include 1 mg/kg body weight or 3 mg/kg body weight via intravenous administration.

A “therapeutically effective amount” or “therapeutically effective amount” of an antibody or ADC or pharmaceutical salts thereof of the invention preferably results in a decrease in severity of disease symptoms, an increase in frequency and/or duration of disease symptom-free periods, prevention or reduction of likelihood of impairment or disability due to the disease affliction, or inhibition or delaying of the progression of disease. For example, for the treatment of tumor-bearing subjects, a “therapeutically effective amount” of an antibody composition may inhibits tumor growth by at least about 20%, more preferably by at least about 40%, even more preferably by at least about 60%, and still more preferably by at least about 80% relative to untreated subjects.

### EXAMPLES

### 1. Generation of 27B4H43L3 (Reference) and BH73 Antibody

Briefly, Balb/C female mice (age 6 to 8-week-old) were immunized with recombinant human B7-H3 antigen and spleen lymphocytes of the mice with the highest titer electrofused with SP2/0 myeloma cells. B7-H3 ELISA was performed to screen for positive binders, and clonal selection was carried out through multiple rounds of subcloning and expansion. Clone 27B4 was identified as one of the highest binders and selected for gene cloning. Humanization through CDR grafting was carried out and B7-H3 binding activity was confirmed a humanized clone, 27B4H43L3, which was made into a huIgG1 format. The heavy chain and light chain sequences for this reference antibody are set forth in SEQ ID NO: 3 and SEQ ID NO: 5, respectively.

An example modified antibody of the present disclosure, BH73 antibody, was generated with the variable sequences of that of 27B4H4L3 antibody, CH1 domain of IgG2, a hinge sequence comprising EPPKSDCKTKTVECPPCP (SEQ ID NO: 12), and IgG1 Fc with LALA mutation (SEQ. L234A, L235A). It was made for the purpose to generate ADC containing two drug payloads at specific locations (site-specific conjugation). The heavy chain and light chain sequences for this clone BH73 are set forth in SEQ ID NO: 4 and SEQ ID NO: 5, respectively.

### 2. Expression and Purification of the 27B4H4L3 and BH73 Antibodies

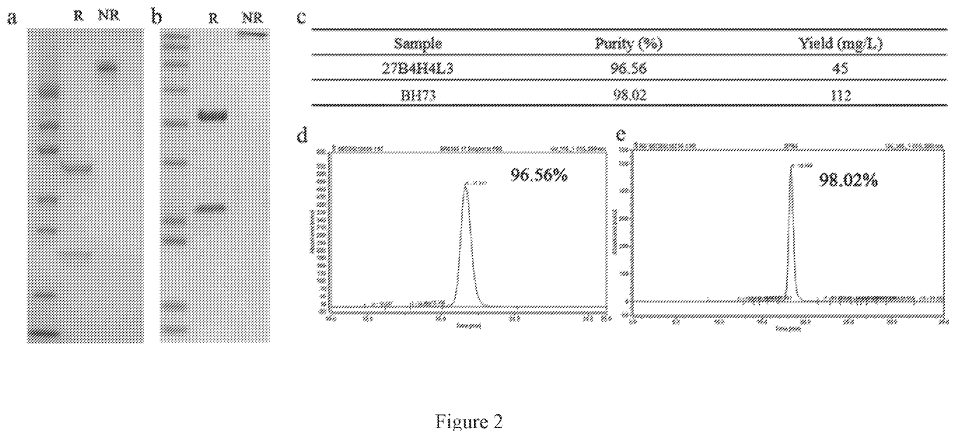

For the expression of the 27B4H4L3 and BH73 antibodies, codon optimization and gene synthesis were performed for better production in HEK293 cells. Full-length heavy chain and light chain DNA were each cloned into a separate pcDNA3 plasmid. HEK293 cell transient transfection of the paired plasmids and one-step protein A purification was used to prepare enough proteins for testing. Antibodies made in this format expressed well with decent yield and could be purified in high purity with one step protein A purification process (FIG. 2).

### 3. Conjugation of 27B4H4L3 Antibody and BH73 Antibody to Generate ADCs

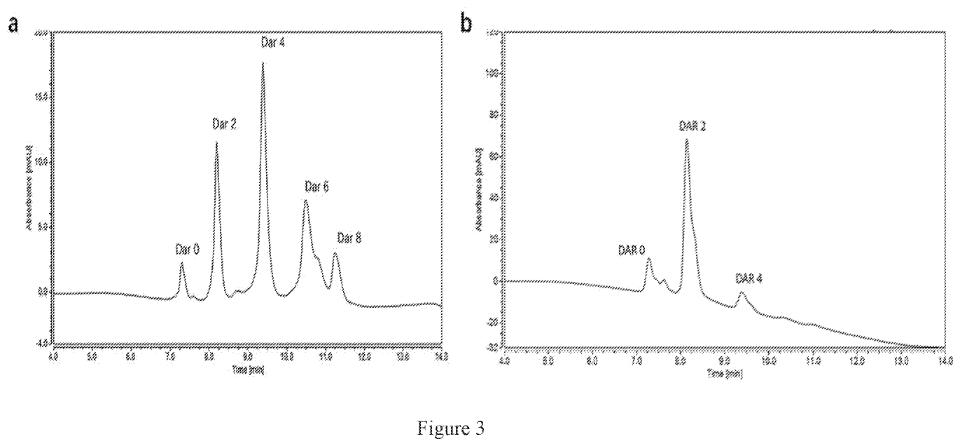

To generate drug conjugates, TCEP was added to a solution containing a purified antibody at neutral pH. Under mild reduction conditions (TCEP: mAb=1-3, neutral pH, room temperature for <240 min), inter-chain disulfide bonds of an antibodies were partially reduced. Drug-linker (MC-Val-Cit-PAB-MMAE) in DMA was added and allowed to react with antibody to obtain desired drug-to-antibody ratio (DAR). To characterize the ADCs, hydrophobic interaction chromatography (HIC) was performed for the evaluation of drug distribution and molar ratio of drug and antibody in ADCs, and drug in ADCs and the representative HIC chromatograms are presented in FIG. 3. Under these mild reduction/conjugation conditions, the ADCs made of 27B4H4L3 contained main peaks of 27B4H4L3 linked to 2, 4, and 6 drug molecules (DAR2, DAR4, DAR6, respectively). The minor peaks are 27B4H4L3 (DAR0) and 27B4H4L3 linked to 8 drug molecules (DAR8) (FIG. 3a). The ADCs made of BH73 antibody were predominantly made of DAR2 ADC species, and the minor peaks are BH73 (DAR0) and BH73 linked to 4 drug molecules (DAR4) (FIG. 3b).

4. Measurement of B7-H3 Binding Activities of Antibodies and their Corresponding ADCs

ELISA assay was used to exam and compare the B7-H3 binding capabilities between antibodies (27B4H4L3 and BH73) and their corresponding ADCs (27B4H4L3-MMAE, BH73-MMAE). Human B7-H3 proteins (1000 ng/mL) were coated onto 96-well plates and the plates were incubated at 4° C. overnight. Diluted samples were then transferred to B7-H3-coated plates and incubated at room temperature for 1.5 h. Using HRP-labeled goat anti-human IgG Fc antibody (Sigma, A0170) as a detection agent and TMB for colorimetric reaction, the plates read at 450/650 nm for absorbance on Microplate Reader (Molecular Devices, SpectraMax 190) and data analysis was performed using a dose response curve format four parameters logistic model.

The results in FIG. 4 and Table 1 showed that BH73, modified from 27B4H4L3, binds B7-H3 with similar EC₅₀ with that of 27B4H4L3; and the ADCs (27B4H4L3-MMAE, and BH73-MMAE) made of 27B4H4L3 antibody or BH73 antibody also had a similar binding activity to B7-H3 as compared with their corresponding naked antibodies, suggesting that payload conjugation did not affect target binding capability of ADCs.

TABLE 1
EC₅₀ of B7-H3 binding activities with 27B4H4L3
antibody, BH73 antibody, 27B4H4L3-Eribulin,
27B4H4L3-MMAE and BH73-Eribulin.
EC₅₀ (ng/ml)

| | 27B4H4L3- | | BH73- |
|---|---|---|---|
| 27B4H4L3 | MMAE | BH73 | MMAE |
| 25.66 | 43.97 | 23.15 | 20.43 |

### 5. Measurement of Binding of Antibodies and ADCs to B7-H3 Expressing Cells

Screening for B7-H3-expressing cells were performed using FACS with 27B4H4L3 antibody and MFI of 27B4H4L3 to those cells shown in Table 2. FACS assay was also used to exam and compare the binding capabilities of the 27B4H4L3 antibody, BH73 antibody, 27B4H4L3-MMAE and BH73-Eribulin to B7-H3-expressing cells. To perform the assay, target cells and samples were incubated at 4° C. for 1 h. After wash, samples and secondary antibody (goat pAb to human IgG (FITC) (Abcam, ab97224)) were incubated. FACS was performed on low cytometer (BD, Accuri C6 Plus) and mean fluorescence intensity (MFI) was reported. Three cell lines (U87MG, BT-474 and HT-29) representing B7-H3 high- and low-expressing cells were used to compare binding capacities of different antibody constructs and ADCs. BH73 antibody, had similar MFI levels of those of 27B4H4L3 antibody on all tested cells; the different forms of ADCs (27B4H4L3-MMAE, and BH73-MMAE) also had similar MFI levels (Table 3), suggesting that their B7-H3 binding activities were not affected by either engineering or conjugation.

TABLE 2
B7-H3 expressing cells used (B7-H3 expression
levels were measured by FACS using
27B4H4L3 antibody and MFIs were reported).

| Cell line (tumor type) | 27B4H4L3(MFI) |
|---|---|
| U87MG (Glioma) | 2.79 × 10⁵ |
| BT-474 (Breast) | 1.47 × 10⁵ |
| N87 (Stomach) | 8.38 × 10⁴ |
| H1299 (Lung) | 7.77 × 10⁴ |
| MDA-MB-231 (Breast) | 7.61 × 10⁴ |
| HT29 (Colon) | 7.26 × 10⁴ |
| A431 (Skin) | 4.82 × 10⁴ |
| NUGC3 (Stomach) | 3.31 × 10⁴ |
| MDA-MB-468 (Breast) | 2.79 × 10⁴ |
| SW480 (Colon) | 2.68 × 10⁴ |
| SW620 (Colon) | 2.49 × 10⁴ |
| NCI-H929 (Marrow) | 5.14 × 10³ |

TABLE 3
MFI of the 27B4H4L3 antibody, BH73 antibody, 27B4H4L3-MMAE,
27B4H4L3-Eribulin and BH73-Eribulin to B7-H3
expressing cells (U87MG, BT474 and HT29).

| Cell line | | 27B4H4L3- | | |
|---|---|---|---|---|
| (tumor type) | 27B4H4L3 | MMAE | BH73 | BH73-MMAE |
| U87(Glioma) | 3.14 × 10⁵ | 2.9 × 10⁵ | 3.33 × 10⁵ | 3.21 × 10⁵ |
| BT-474 (Breast) | 1.26 × 10⁵ | 1.26 × 10⁵ | 1.33 × 10⁵ | 1.38 × 0⁵ |
| HT-29 (Colon) | 6.95 × 10⁴ | 6.53 × 10⁴ | 7.15 × 10⁴ | 7.13 × 10⁴ |

### 6. Cytotoxicity of ADCs to B7-H3 Expressing Cells

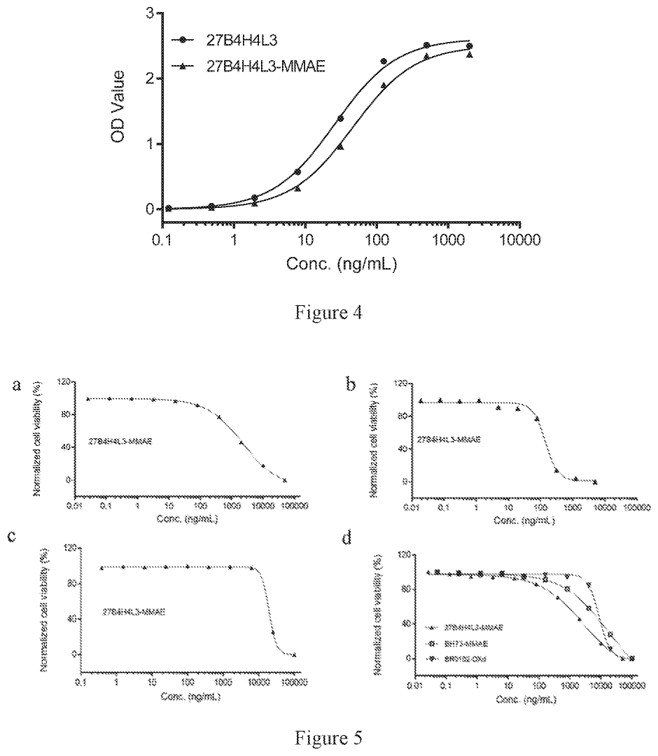

The cytotoxicity of the ADCs comprising antibody portion of 27B4H4L3 or BH73 were evaluated and compared in in vitro cytotoxicity assay to multiple cell lines with different levels of B7-H3 expression. To perform the assay, target cells were seeded into a 96-well flat-bottom tissue culture plate at an optimized cell density for each cell line and incubated at 37° C., 5% CO₂ overnight. Serial dilutions of ADCs samples were transferred to cell plate and the assay plates were incubated for a defined period of time (3-7 days depend on cell lines) for optimal killing. Data analysis was performed using a dose response curve by four parameters logistic model. As shown in FIG. 5, 27B4H4L3-MMAE and BH73-MMAE showed cytotoxicity activities to B7-H3-expresing cancer cells.

### 7. In Vivo Animal Models and Treatment

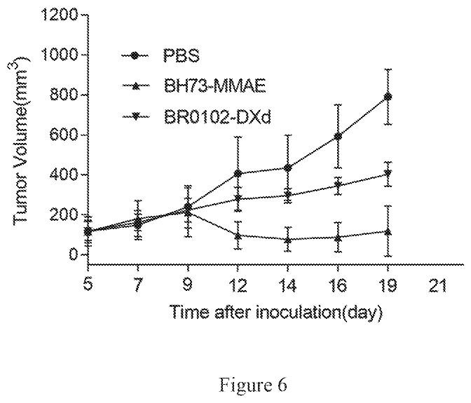

The in vivo antitumor activities of ADCs comprising antibody portion of BH73 were assessed and compared with that of BR0102-DXd ADC (WO_2020_130125_A1). In HT29 colorectal cancer xenografts model established in nude mice. HT29 cells were implanted into the back flank of athymic nude mice. Tumor-bearing mice were treated with vehicle (control) or ADCs solution once through tail vein injection. ADCs were given at the dose of 20 mg/kg. The results shown in FIG. 6 demonstrated that BH73-MMAE treatment was effective in tumor suppression in HT29 model while BR0102-DXd treatment at the same dose levels was only minimally effective.

### REFERENCES

[1]. Seaman S, Stevens J, et al., Cancer Cell, 2007, 11, 539-554.
[2]. Brunner A, Hinterholzer, S, et al., Gynecol. Oncol., 2012, 124, 105-111.
[3]. Huang C, Zhou L, et al., Oncol. Rep., 2016, 35, 2183-2190.
[4]. Arigami T, Narita N, et al., Ann. Surg., 2010, 252, 1044-1051.
[5]. Chen J T, Chen C H, et al., Proc. Nal. Acad. Sci., 2015, 112, 13057-13062.
[6]. Crispen P L, Sheinin Y, et al., Clin. Cancer Res., 2008, 14, 5150-5157.
[7]. Brunner A, Hinterholzer S, et al., Gynecol. Oncol., 2012, 124, 105-111.
[8]. Qin X, Zhang H, et al., Onco. Targets Ther., 2013, 6, 1667-1673.
[9]. Zang X, Sullivan P S, et al., Mod. Pathol., 2010, 23, 1104-1112.
[10]. Loo D, Alderson R F, et al., Clin. Cancer Res., 2012, 18, 3834-3845.
[11]. Ahmed M, Cheng M, et al., J. Biol. Chem., 2015, 290, 30018-30029.
[12]. Modak S, Kramer K, et al., Cancer Res., 2001, 61, 4048-4054.
[13]. Chen Z Z, Wang L P, et al., Curr. Pharm. Biotechno., 2016, 17, 1324-32.
[14]. Ogitani Y, Abe Y, et al., Bioorg. Med. Chem. Lett., 2016, 26, 5069-72.
[15]. Wang G, Wu Z, et al., Oncolarget, 2016, 7, 24888-98.

All patents and non-patent literature references described herein are incorporated by reference herein in their entireties.

While the invention has been described above in connection with one or more embodiments, it should be understood that the invention is not limited to those embodiments, and the description is intended to cover all alternatives, modifications, and equivalents, as may be included within the spirit and scope of the appended claims.

What is claimed is:

1. An isolated antibody or an antigen-binding portion thereof, comprising:
two heavy chains each comprising:(a1) a heavy chain hinge region comprising the amino acid sequence set forth in any of SEQ ID NOs: 12-24;
(a2) a heavy chain variable domain comprising a CDR1 region, a CDR2 region, and a CDR3 region comprising the amino acid sequences of SEQ ID NO: 6, SEQ ID NO: 7, and SEQ ID NO: 8, respectively, and
two light chains each comprising:
(a3) a light chain variable domain comprising a CDR1 region, a CDR2 region, and a CDR3 region comprising the amino acid sequences of SEQ ID NO: 9, WAS, and SEQ ID NO: 10, respectively.

2. The antibody or the antigen-binding portion thereof, of claim 1, wherein the antibody specifically binds to human B7-H3 protein.

3. The antibody or the antigen-binding portion thereof, of any of claims 1-2, wherein the amino acid sequence comprised in the heavy chain hinge region is SEQ ID NO: 12.

4. The antibody or the antigen-binding portion thereof, of any of claims 1-3, wherein each of the heavy chains further comprises: a human CH1 domain located upstream of and connected to the hinge region, the CH1 domain comprising a cysteine at the position of 142 according to the IMGT numbering scheme.

5. An isolated antibody or an antigen-binding portion thereof, comprising:
two heavy chains each comprising:
(a) a hinge region comprising an amino acid sequence of: —(X₁)—C—(X₂)—CPPCP—, wherein X₁ is a polypeptide segment having 0-7 amino acid residues each independently selected from any amino acid residue that is not a cysteine residue, and X₂ is a polypeptide segment having 2-7 amino acid residues each independently selected from any amino acid residue that is not a cysteine residue;
(b) a human CH1 domain located upstream of and connected to the hinge region, the CH1 domain comprising a cysteine at the position of 142 according to the IMGT numbering scheme;
wherein the antibody specifically binds to human B7-H3 protein.

6. The antibody or the antigen-binding portion thereof, of claim 5, wherein the amino acid sequence of comprised in the hinge region is selected from the group consisting of SEQ ID NOs: 12-24.

7. The antibody or the antigen-binding portion thereof, of any of claims 5-6, wherein the amino acid sequence comprised in the heavy chain hinge region is SEQ ID NO: 11.

8. The antibody or the antigen-binding portion thereof, of any of claims 5-6, wherein the amino acid sequence comprised in the heavy chain hinge region is SEQ ID NO: 12.

9. The antibody or the antigen-binding portion thereof, of any of the claims 5-8, further comprising two kappa light chains each paired with one of the heavy chains.

10. The antibody or the antigen-binding portion thereof, of any of claims 4-9, wherein the CH1 domain of the antibody has the same sequence as that of the CH1 domain of a native human IgG2, IgG3, or IgG4 subclass antibody.

11. The antibody or the antigen-binding portion thereof, of any of claims 4-9, wherein the CH1 domain of the antibody has the sequence of that of the CH1 domain of a native human IgG1 antibody with the mutation S142C according to the IMGT numbering scheme.

12. The antibody or the antigen-binding portion thereof, of any of claims 4-9, wherein each of the heavy chains further comprises a Fc domain of a native human IgG1, IgG2, IgG3, IgG4 subclass antibody downstream of and connected to the hinge region, wherein the Fc domain optionally includes one or more substitutions.

13. The antibody or the antigen-binding portion thereof, of any of the foregoing claims, wherein each of the heavy chains comprises a variable domain comprising the amino acid sequence set forth in SEQ ID NO: 1.

14. The antibody or the antigen-binding portion thereof, of any of the foregoing claims, wherein each of the heavy chains comprises an amino acid sequence set forth in SEQ ID NO: 3.

15. An antibody-drug conjugate (ADC) or a pharmaceutically acceptable salt thereof, comprising:
an antibody of any of the claims 1-12 conjugated to a cytotoxic drug by a chemical linker.

16. An antibody-drug conjugate (ADC) or a pharmaceutically acceptable salt thereof, comprising:
(A) an isolated antibody, or an antigen-binding portion thereof, comprising:
(a1) a heavy chain hinge region comprising the amino acid sequence set forth in SEQ ID NO:11; and
(a2) a heavy chain variable domain comprising a CDR1 region, a CDR2 region, and a CDR3 region comprising the amino acid sequences of SEQ ID NO:6, SEQ ID NO:7, and SEQ ID NO:8, respectively, and
(a3) a light chain variable domain comprising a CDR1 region, a CDR2 region, and a CDR3 region comprising the amino acid sequences of SEQ ID NO:9, WAS, and SEQ ID NO:10, respectively;
(B) a cytotoxic drug, wherein the isolated antibody or an antigen-binding portion thereof is conjugated to the cytotoxic drug by a chemical linker.

17. The ADC or a pharmaceutically acceptable salt thereof, of any of claims 15 or 16, wherein the cytotoxic drug is selected from the group consisting of monomethyl auristatin E (MMAE), monomethyl auristatin F (MMAF), auristatin E, auristatin F, maytansine DM1 and DM4, maytansinol, sandramycin, pyrrolobenzodiazepine, pyrrolobenzodiazepine dimer, anthracyclines, calicheamicin, dolastatin 10, duocarmycin, doxorubicin, thailanstatin A, uncialamycin, amanitins, ricin, diphtheria toxin, ¹³¹I, interleukins, tumor necrosis factors, chemokines, irinotecan (SN38), exatecan, eribulin, and nanoparticles.

18. The ADC or a pharmaceutically acceptable salt thereof, of any of claims 15 or 16, wherein the chemical linker comprises a portion that is selected from the group consisting of 6-maleimidocaproyl (MC), maleimidopropionyl (MP), valine-citrulline (Val-Cit), alanine-phenylalanine (ala-phe), p-aminobenzyloxycarbonyl (PAB), 6-maleimidocaproyl-Val-Cit-p-aminobenzyloxycarbonyl (MC-Val-Cit-PAB), Mal-PEGₙ-Val-Cit-PAB (n=1-20), Mal-amido-PEGₙ-Val-Cit-PAB (n=1-20), MC-Gly-Gly-Phe-Gly, Phe-Lys(Fmoc)-PAB, Aloc-D-Ala-Phe-Lys(Aloc)-PAB-PNP, Boc-Phe-(Alloc)Lys-PAB-PNP, and perfluorophenyl 3-(pyridine-2-yldisulfanyl) propanoate.

19. A pharmaceutical composition comprising: an isolated antibody or an antigen binding portion thereof of any of claims 1-12, or an ADC of pharmaceutically acceptable salt thereof, of claims 15-18, and a pharmaceutical acceptable carrier.

20. A method of treating cancer in a human subject, comprising administering an effective amount of the pharmaceutical composition of claim 18.

21. The method of claim 20, wherein the cancer is associated with overexpression of B7-H3 protein.

22. The method of claim 20, wherein the cancer is selected from the group consisting of a cancer of head and neck, skin, colon, kidney, glioblastoma, glioma, thyroid, mesothelioma, melanoma, pancreas, lung, breast, ovary, prostate, and bladder.


SEQUENCE LISTING (24 sequences)

SEQ ID NO: 1 - protein, 120 aa, Artificial Sequence
  VH of 27B4H4L3 and BH73
EVQLVQSGAEVKKPGESLKISCKASGYTFTTYWIGWVRQMPGKGLEWMGIIYPGDGGSRYSPSFQGQVTISADKSISTAYLQWSSLKASDTAMYYCARGGNYISFPMYYWGQGTLVTVSS

SEQ ID NO: 2 - protein, 107 aa, Artificial Sequence
  VL of 27B4H4L3 and BH73
EIVMTQSPATLSVSPGERATLSCKASRGVSTALAWYQQKPGQAPRLLIYWASTRATGIPARFSGSGSGTEFTLTISSLQSEDFAVYYCQQHYDTPYTFGQGTKLEIK

SEQ ID NO: 3 - protein, 450 aa, Artificial Sequence
  Heavy Chain of 27B4H4L3
EVQLVQSGAEVKKPGESLKISCKASGYTFTTYWIGWVRQMPGKGLEWMGIIYPGDGGSRYSPSFQGQVTISADKSISTAYLQWSSLKASDTAMYYCARGGNYISFPMYYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELVGGPSVFLLPPKPKDTLMISRTPEVTCVVVDVSHEDPEVKFNWYVDGVEVHNAKTKPPEEQYNSTLRVVSVLTVLHQDWLNGKEYKCKVSNKALPAPIEKTISKAKGQPREPQVYTLPPSREEMTKNQVSLTCLVKGFYPSDIAVEWESNGQPENNYKTTPLVLDSDGSFFLYSKLTVDKSRWQQGNVFSCSVMHEALHNHYTQKSLSLSPGK

SEQ ID NO: 4 - protein, 453 aa, Artificial Sequence
  Heavy Chain of BH73
EVQLVQSGAEVKKPGESLKISCKASGYTFTTYWIGWVRQMPGKGLEWMGIIYPGDGGSRYSPSFQGQVTISADKSISTAYLQWSSLKASDTAMYYCARGGNYISFPMYYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVEPPKSDCKTKTVECPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 214 aa, Artificial Sequence
  VL-CL (Human Kappa) of 27B4H4L3 and BH73
EIVMTQSPATLSVSPGERATLSCKASRGVSTALAWYQQKPGQAPRLLIYWASTRATGIPARFSGSGSGTEFTLTISSLQSEDFAVYYCQQHYDTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 8 aa, Artificial Sequence
  Heavy Chain CDR1 of BH73 (and 27B4H4L3)
GYTFTTYW

SEQ ID NO: 7 - protein, 8 aa, Artificial Sequence
  Heavy Chain CDR2 of BH73 (and 27B4H4L3)
IYPGDGGS

SEQ ID NO: 8 - protein, 13 aa, Artificial Sequence
  Heavy Chain CDR3 of BH73 (and 27B4H4L3)
ARGGNYISFPMYY

SEQ ID NO: 9 - protein, 6 aa, Artificial Sequence
  Light Chain CDR1 of BH73 (and 27B4H4L3)
RGVSTA

SEQ ID NO: 10 - protein, 9 aa, Artificial Sequence
  Light Chain CDR3 of BH73 (and 27B4H4L3)
QQHYDTPYT

SEQ ID NO: 11 - protein, 15 aa, Artificial Sequence
  Hinge Sequence of Heavy Chain of 27B4H4L3
EPKSCDKTHTCPPCP

SEQ ID NO: 12 - protein, 18 aa, Artificial Sequence
  Hinge Sequence of Heavy Chain of BH73
EPPKSDCKTKTVECPPCP

SEQ ID NO: 13 - protein, 10 aa, Artificial Sequence
  Hinge Sequence of Heavy Chain of Other Examples
CKTHTCPPCP

SEQ ID NO: 14 - protein, 11 aa, Artificial Sequence
  Hinge Sequence of Heavy Chain of Other Examples
DKTCHTCPPCP

SEQ ID NO: 15 - protein, 12 aa, Artificial Sequence
  Hinge Sequence of Heavy Chain of Other Examples
ERKSCVECPPCP

SEQ ID NO: 16 - protein, 15 aa, Artificial Sequence
  Hinge Sequence of Heavy Chain of Other Examples
EPKSDCKTHTCPPCP

SEQ ID NO: 17 - protein, 15 aa, Artificial Sequence
  Hinge Sequence of Heavy Chain of Other Examples
EPKSDKCTHTCPPCP

SEQ ID NO: 18 - protein, 17 aa, Artificial Sequence
  Hinge Sequence of Heavy Chain of Other Examples
EPKSDCKTHTVECPPCP

SEQ ID NO: 19 - protein, 15 aa, Artificial Sequence
  Hinge Sequence of Heavy Chain of Other Examples
EPKSDCKTVECPPCP

SEQ ID NO: 20 - protein, 17 aa, Artificial Sequence
  Hinge Sequence of Heavy Chain of Other Examples
EPKSDKCTHTVECPPCP

SEQ ID NO: 21 - protein, 18 aa, Artificial Sequence
  Hinge Sequence of Heavy Chain of Other Examples
EPPKSCDKTHTVECPPCP

SEQ ID NO: 22 - protein, 18 aa, Artificial Sequence
  Hinge Sequence of Heavy Chain of Other Examples
EPPKSDCKTHTVECPPCP

SEQ ID NO: 23 - protein, 19 aa, Artificial Sequence
  Hinge Sequence of Heavy Chain of Other Examples
EPPPKSCDKTHTVECPPCP

SEQ ID NO: 24 - protein, 20 aa, Artificial Sequence
  Hinge Sequence of Heavy Chain of Other Examples
EPPPPKSCDKTHTVECPPCP